(19) United States
(12) Patent Application Publication
(10) Pub. No.: US 2023/0261885 A1
(43) Pub. Date: Aug. 17, 2023

(54) CREATION AND USE OF LEDGER FILES

(71) Applicant: GEMVIZ, LLC, Winona Lake, IN (US)

(72) Inventors: Jody L. Claypool, Warsaw, IN (US); Vinay Tikka, Winona Lake, IN (US)

(21) Appl. No.: 18/110,282

(22) Filed: Feb. 15, 2023

Related U.S. Application Data
(60) Provisional application No. 63/310,227, filed on Feb. 15, 2022.
(60) Provisional application No. 63/310,280, filed on Feb. 15, 2022.

(51) Int. Cl. H04L 9/00; H04L 9/32

(52) U.S. Cl. CPC H04L 9/50; H04L 9/3297

(57) ABSTRACT

The creation and use of ledger files can support the association of an asset in real-space to a virtual/digital form as things change with the asset over time. An example process can include receiving a request to create a ledger file for an asset related to a physical entity and having associated initial attribute data and a digital model; creating the ledger file, the ledger file being identified by a first object identifier and storing the associated initial attribute data and the digital model; generating a first security tag for the digital model comprising information of a first block hash to the digital model and a first timestamp; generating a second security tag for the initial attribute data comprising information of a second block hash to the initial attribute data and a second timestamp; and updating an index of the ledger file by storing the first and second security tags.

### CROSS-REFERENCE TO RELATED APPLICATION(S)

This application claims the benefit of U.S. Provisional Application Ser. No. 63/310,227, filed Feb. 15, 2022, and U.S. Provisional Application Ser. No. 63/310,280, filed Feb. 15, 2022.

### BACKGROUND

Virtual reality describes an immersive simulated experience. Items in a virtual reality environment may be designed to appear as realistic as possible or, even if presented as something not possible or that does not exist in the real world, have features and functionality that people are familiar with from the real world. With the continued interest in creating a “metaverse” where people can communicate with each other, perform activities, and conduct transactions in a virtual and augmented reality environment, there is a need for technologies that can support the association between the real physical entity (e.g., person, thing) and the virtual representation of that entity, especially as things change with respect to the real physical entity over time.

### BRIEF SUMMARY

The creation and use of ledger files are described. The described technology supports the association of an asset in real-space to a virtual/digital form as things change with the asset over time. This allows for ease of tracking an entity's origination, the development process used to make it, and who had access to the entity during the entity's useful life. Real world attributes are able to be attributed to a virtual asset.

The creation and use of the ledger files can include receiving a request to create a ledger file for an asset related to a first physical entity having associated initial attribute data and a digital model; creating the ledger file for the asset related to the first physical entity, the ledger file being identified by a first object identifier and storing the associated initial attribute data and the digital model, wherein the associated initial attribute data corresponds to a detail associated with the asset related to the first physical entity, wherein the ledger file stores the associated initial attribute data with properties providing a record of information about the asset related to the first physical entity or the detail associated therewith; generating a first security tag for the digital model, the first security tag comprising information of a first block hash to the digital model and a first timestamp; generating a second security tag for the initial attribute data, the second security tag comprising information of a second block hash to the initial attribute data and a second timestamp; and updating an index of the ledger file by storing the first security tag and the second security tag. In some cases, the creating and updating of the ledger file is for an asset related to a medical device. In some cases, the creating and updating of the ledger file is for an asset related to a patient.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1 illustrates an example conceptual representation of a ledger file.

FIG. 2A illustrates an example process flow for creating and updating a ledger file according to certain embodiments of the invention.

FIG. 2B illustrates an example process flow of using a ledger file according to certain embodiments of the invention.

FIG. 3 illustrates an example process flow for creating and updating a ledger file for a medical device according to certain embodiments of the invention.

FIGS. 4A-4C illustrate an example scenario of creating and updating a ledger file for an asset related to a medical device.

FIG. 5 illustrates an example process flow for creating and updating a ledger file for an asset related to a patient according to certain embodiments of the invention.

FIGS. 6A-6C illustrate an example scenario of creating and updating a ledger file for an asset related to a patient.

FIG. 7 and FIG. 8 illustrate example scenarios relating to a patient and a medical device according to certain embodiments of the invention, where FIG. 7 illustrates an example scenario of updating a ledger file for an asset related to the patient to include the medical device after the medical device has been implanted in the patient and FIG. 8 illustrates an example scenario of accessing data associated with the medical device.

FIG. 9 illustrates a correlation between a virtual and physical entity during attribution of Object ID while the entity moves from origin to end of life according to certain embodiments of the invention.

FIG. 10 illustrates an example patient care platform with a ledger file integration in accordance with the subject disclosure.

FIG. 11 illustrates an example interface of a patient care platform displaying a surgeon dashboard.

FIGS. 12A and 12B illustrate example interfaces of a patient care platform displaying a selected patient view.

### DETAILED DESCRIPTION

The creation and use of ledger files are described. The described technology supports the association of an asset in real-space to a virtual/digital form as things change with the asset over time. The association can be performed synchronously such that as information is collected about the asset (e.g., by sensors, manual entry of information, etc.), the virtual/digital information is also updated.

A ledger file format, referred to herein as an EOX file format, is used to track changes/updates to a digital model/asset in its entire life cycle, following the changes and updates of the corresponding physical asset and/or physical entity to which the asset is related. An asset is a useful or valuable thing, person, or quality. A physical entity is a thing with a distinct and independent existence, which has a presence that is tangible. An asset related to a physical entity is not required to be of material existence and can include concepts and other abstractions. The described EOX file format can be used to support the association between the real physical asset and the virtual representation of that asset as things change over time.

Through the use of the ledger file, it is possible for digital assets to track various attributes of the physical world representation of those digital assets. As the physical asset/physical world representation goes through its life cycle (e.g., from manufacturing to utilization to retirement), the ledger file not only tracks any changes to the attributes but also maintains the validity of the changes through an update index of the ledger file.

Application of the EOX file and its tracing data through the life cycle of an asset is helpful for medical devices, implants, pharmaceuticals, nanoceuticals, sports gear, and many other physical entities. For example, the described technology enables ease of tracking of a medical device's origination, the development process used to make the medical device, and who had access to the medical device during its useful life. This provides transparency to the change of the entity's attributes between the physical and the virtual.

Advantageously, product developers can use files that are typically involved in the development of product, such as CAD files, with the associated metadata in the creation of a virtual reality compatible virtual asset. This can be thought of as creating an identical virtual and physical asset. This can improve the user experience and the reliability of the virtual asset in the virtual world and predictive modeling algorithms, due to quantification of variables.

The described technology can also be helpful in a variety of healthcare settings. The described technology can integrate with existing healthcare IT systems and can consolidate all patient data to individualize patient care, thus allowing for an increase in regular patient engagement on their health journey, monitoring of concerns and flagging of issues, and deepened clinical insights and patient interactions. encrypts and decentralizes data to address privacy and security concerns while enabling actionable insights.

There is currently a wealth of information about patients that is readily available to practitioners but not being used to make the proper diagnosis and treatment plan for an individual patient. Indeed, limited information access, time, and resources result in less personalized patient treatment, reduced engagement, and sub-optimal outcomes. For example, a hospital or practitioner may have to use multiple interfaces to access all of the patient's specific data resulting in the practitioner not having enough time to reference all the patient specific data.

Further, patients are often frustrated by long waits, repetitive questions, and inconsistent interactions with a hospital or practitioner. In some cases, patients may lie to a practitioner about diet, activity level, substance use, etc. when meeting with a practitioner or forget important information discussed during the visit. These issues may lead to an improper or unclear diagnosis. For example, a patient may not have a clear understanding of what their diagnosis or what their treatment path is. Advantageously, the generation and use of EOX files enables a stronger connection between digital information and the physical entity (person or thing) over time.

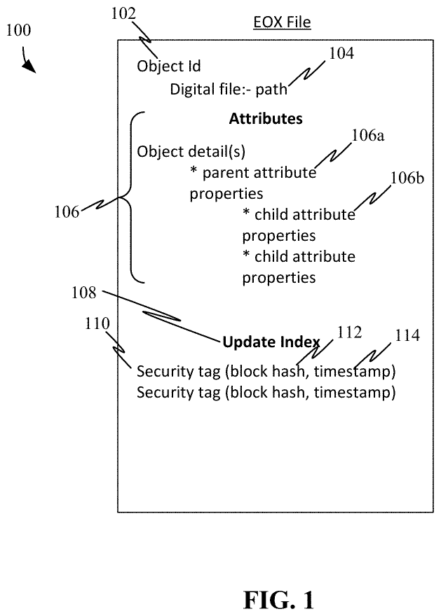

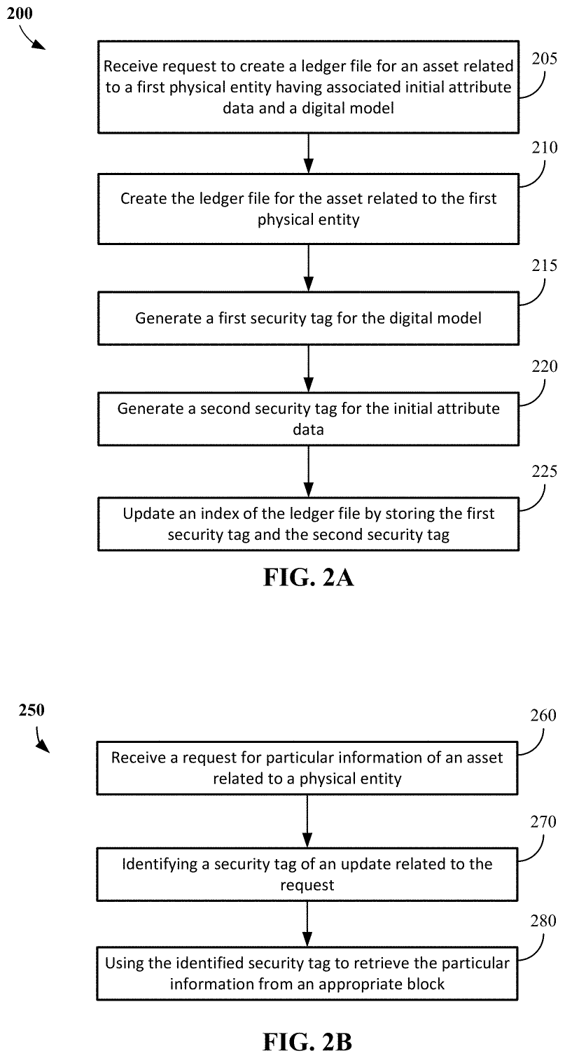

FIG. 1 illustrates an example representation of a ledger file and FIG. 2A illustrates an example process flow for creating and updating a ledger file according to certain embodiments of the invention.

Referring to FIG. 1, a data structure of a ledger file 100 includes an object identifier 102 that identifies the ledger file for an asset related to a physical entity; a digital model 104 of the asset related to the physical entity; attributes 106, each attribute corresponding to a detail associated with the asset related to the physical entity and having properties providing a record of information about the asset related to the physical entity or the detail associated therewith; and an updates index 108 for storing security tags 110, each security tag having information including a block hash 112 and a timestamp 114.

The object identifier 102 identifies the ledger file for an asset related to a physical entity in the physical world. This identifier can be considered a routing number that associates the virtual asset with its respective asset in the physical world. The object identifier 102 represents the parent entity to which attributes can be applied (e.g., the top most element to which any other assets and attributes can be added to or built from). Conceptually, the object identifier can be considered to identify a primary block from which next blocks are added or a root of a tree from which next nodes are connected. A block can be defined as a set/collection of things (e.g., objects, transactions, etc.), where the size is predefined.

The digital model 104 of the asset related to the physical entity can be a 3-D model file, a virtual reality file, an image file, a document file, and the like. In some cases, the ledger file 100 includes the digital model 104 in the form of resource location information (e.g., a file path) of the digital model. In some cases, multiple digital models may be included, for example, of different file types for different visual environments (e.g., virtual reality, augmented reality, conventional display screens).

An attribute 106 corresponds to a detail associated with the asset related to the physical entity. The association may be based on a direct relationship, for example, based on any form of information about the physical entity. In some cases, a loose association of attributed data can be included, allowing for additional connections between entities. Examples of loosely associated attributes include community information (e.g., information from other similar physical entities or other users who have acquired similar entities), funding and regulation information (e.g., insurance/government regulation), and other aspects that may of interest for filtering and sorting of information.

Attributes 106 can include a parent attribute 106a indicating a source of the information or indicative of a category of information; and one or more child attributes 106b of the parent attribute, the one or more child attributes being related to content associated with the source of information or being indicative of an entity in the category of information.

An attribute 106 (e.g., parent attribute 106a or child attribute 106b) can indicate the source of the information, for example, an application or device from which information about the asset and/or physical entity is received or derived.

Attributes 106 can include attribution information indicating a source or creator of the asset related to the physical entity and/or the physical entity itself.

In addition to a block hash 112 and timestamp 114, a security tag 110 can further include what or who is a source of the update. In some cases, the source of the update is an application or device. In some cases, the source of the update indicates the attribute that is updated, for example, when the attribute is an application or device.

The ledger file 100 thus can be composed of blocks where each block stores data about the asset related to the physical entity and its attributes.

The ledger file 100 allows for a means to record data of an entity's development, sale, use, growth, and changes to be recorded in a manner that can easily be converted to information reflected in a virtual reality space or other graphical user interface. The particular activities (and attributes) tracked for an asset depends on the particular asset/physical entity. New attributes can be added to an existing ledger file 100 as well as enable updates to data associated with existing attributes.

The file format described above can enable synchronous association of an asset in real-space to a virtual/digital form as things change with the asset over time. The accumulated information can be stored in a distributed environment or decentralized environment with each successive update referencing the immediate previous location (e.g., using the security tag/block hash and referencing a prior block on the chain).

The ledger file 100 provides a way to logically group things together in a manner that allows for ease of real-time updating/synchronous updating of information in the real world and the digital world.

The organizational structure illustrated in FIG. 1 is for conceptual purposes and the content of the EOX file may be stored in a same or separate storage. In addition, the structure of the EOX file is a logical structure and not necessarily a physical structure. Thus, memory storage configured according the described EOX file need to store the file contiguously.

Referring to FIG. 2A, some or all of process 200 may be executed at, for example, one or more servers as part of services (e.g., a server may include instructions to perform process 200. In some cases, process 200 may be executed at a computing device while in communication with one or more servers.

Process 200 can include receiving (205) a request to create a ledger file, such as ledger file 100 of FIG. 1, for an asset related to a first physical entity having associated initial attribute data and a digital model and creating (210) the ledger file for the asset related to the first physical entity, the ledger file being identified by a first object identifier and storing the associated initial attribute data and the digital model. The associated initial attribute data can correspond to a detail associated with the asset related to the first physical entity and the ledger file can store the associated initial attribute data with properties providing a record of information about the asset related to the first physical entity or the detail associated therewith.

Here, the creation of the ledger file creates a first block having the ObjectId and containing content (the associated initial attribute data and the digital model). The ledger file can include the location (i.e., path) information for the content or be stored with content/digital model itself. Here, the content supports the physical entity to which the asset relates. For example, the asset can be a person which will be represented as an avatar and the physical entity can be a specific person. In such a case, the digital model is a 3D model file and/or a virtual reality (XR) file of the specific person used to generate the avatar of the specific person. As another example, the asset related to the physical entity can be an asset related to a medical device. The asset can be considered a concept or other abstraction holding space for a medical device (e.g., to capture the information and concepts of a life cycle of a medical device from design through use and end of use). In such a case, the digital model can include a CAD file of the medical device (or a file path to the CAD file) generated during a design stage of the medical device, providing a virtual representation of the medical device. Where there is a virtual reality file format representation, the virtual reality file or file path to the virtual reality file can also be included.

Process 200 can further include generating (215) a first security tag for the digital model, the first security tag comprising information of a first block hash to the digital model and a first timestamp; generating (220) a second security tag for the initial attribute data, the second security tag comprising information of a second block hash to the initial attribute data and a second timestamp; and updating (225) an index of the ledger file by storing the first security tag and the second security tag.

In some cases, process 200 can further include receiving second attribute data associated with the asset related to the first physical entity; generating a third security tag for the second attribute data, the third security tag comprising information of a third block hash to the second attribute data and a third timestamp; and updating the index of the ledger file by storing the third security tag.

Process 200 can be performed each time a new ledger file is to be created (e.g., for each physical entity).

In some cases, such as when two physical entities having corresponding ledger files are connected (e.g., a patient having a medical device implant), an attribute can be added to one or both ledger files of the physical entities, indicating a relationship between the first physical entity and a second physical entity and providing a mechanism to access information from that other ledger file (e.g., through a chain of security tags).

Once an EOX file/ledger file is created and during the lifetime of the file in which the file continues to be updated, the information (e.g., attributes) and digital model can be retrieved and used in virtual environments and other means for displaying and interacting with the information.

FIG. 2B illustrates an example process flow of using an EOX file according to certain embodiments of the invention. Information from the ledger file can be retrieved using a corresponding security tag. Authentication credentials, keys, tokens, and other security measures that enable access to the data can be used.

Referring to FIG. 2B, in some embodiments, process 250 includes receiving (260) a request to retrieve particular information of the asset related to a physical entity (e.g., the first physical entity); identifying (270) a corresponding security tag of an update related to the request; and using (280) the identified security tag to retrieve the particular information from an appropriate block. The request to retrieve the particular information of the asset related to the physical entity can include the object identifier and a search query. The search query can be about the asset, the physical entity, a particular detail, an attribute of interest, etc. For the most recent information, the timestamp information of the security tag can be used.

Following the process 250 described in FIG. 2B, an application in which a digital model is being displayed can request particular information about the physical entity from the ledger file. The request for particular information can be referred to as a search query and may be in any suitable format for any suitable corresponding search process as well known in the art. However, the security tags are required to be identified at the ledger file in order to access the information to conduct the search and/or provide results. The results of the query can be the particular information requested or content deemed sufficiently relevant (e.g., having a confidence value above a threshold).

With the security tags and data that are included in the ledger file, it is possible to aggregate the information and generate metrics. For example, based on a search parameter, results of the search (for retrieving particular information from the ledger file) can be evaluated for metrics.

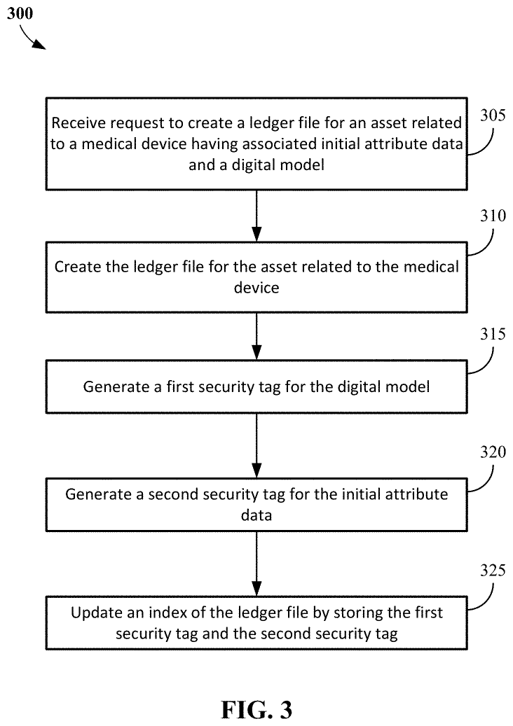

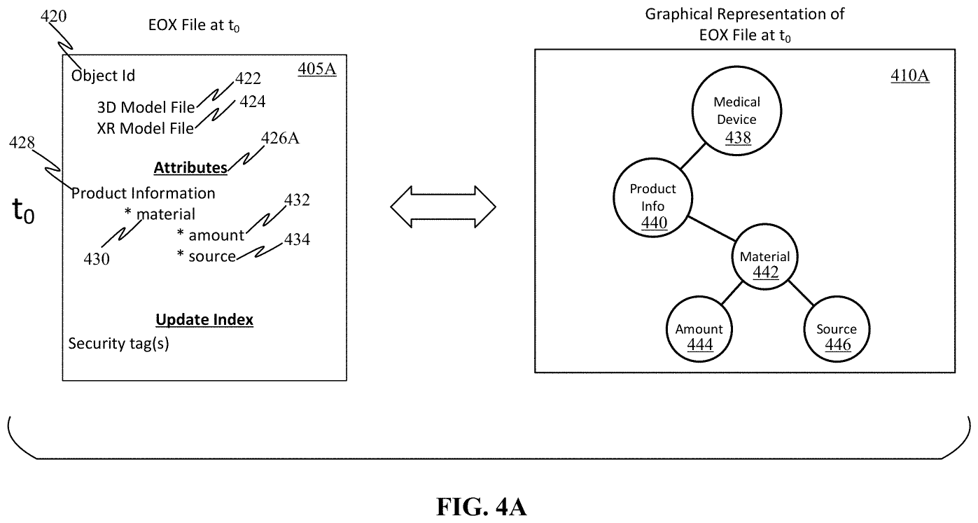

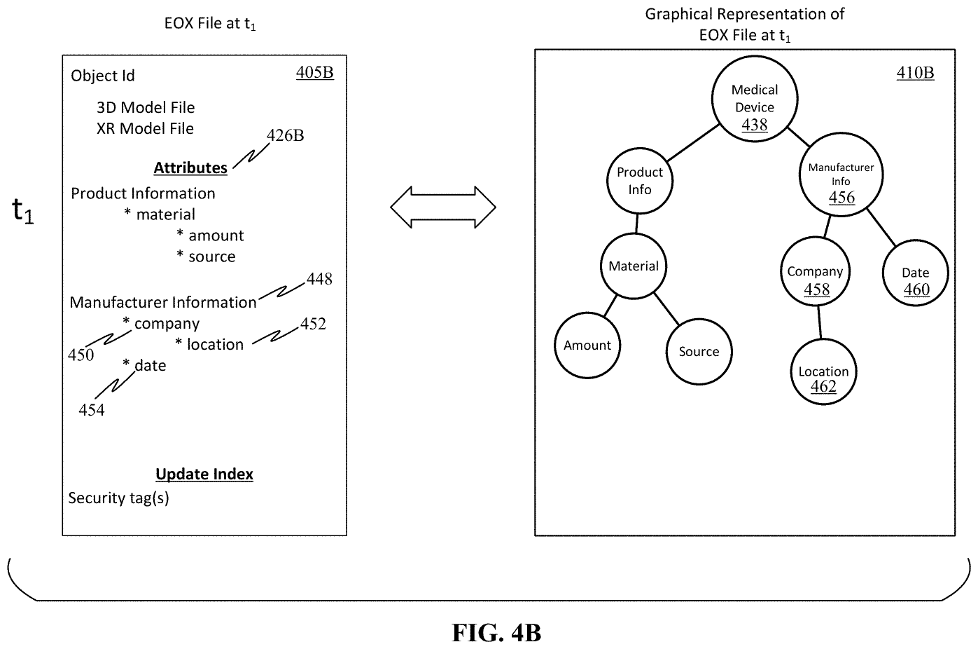

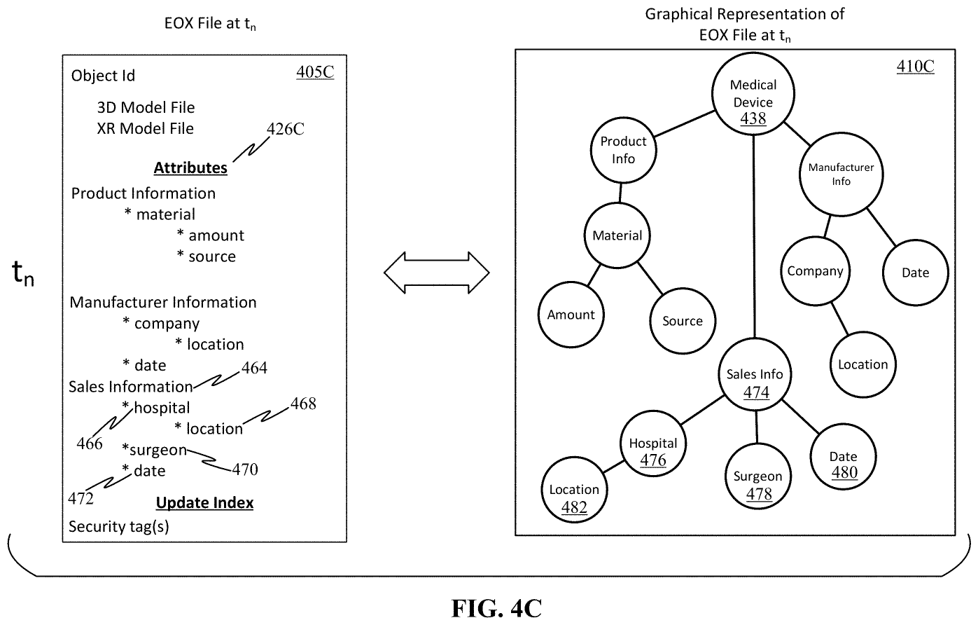

FIG. 3 illustrates an example process flow for creating and updating a ledger file for an asset related to a medical device and FIGS. 4A-4C illustrate an example scenario of creating and updating a ledger file for an asset related to a medical device according to certain embodiments of the invention.

As previously described, the EOX file format can be used to track changes/updates to a digital model/asset in its entire life cycle and follow its corresponding physical entity. Indeed, an EOX file provides a way for digital assets to track various attributes it represents in the physical world. In the illustrative examples of FIG. 3 and FIGS. 4A-4C, the asset is related to a medical device. The asset can be considered a concept or other abstraction holding space for a medical device (e.g., to capture the information and concepts of a life cycle of a medical device from design through use and end of use). As the asset related to the medical device goes through its life cycle from manufacturing to utilization to retirement, an EOX file can act as a ledger to track any changes to the attributes and also maintain the validity of the changes using concepts from the blockchain.

Referring to FIG. 3, some or all of process 300 may be executed at, for example, one or more servers as part of services (e.g., a server may include instructions to perform process 300. In some cases, process 300 may be executed at a computing device while in communication with one or more servers.

Process 300 can include receiving (305) a request to create a ledger file for an asset related to a medical device. The asset related to the medical device includes associated initial attribute data and a digital model. In some cases, the digital model of the asset related to the medical device comprises resource location information (e.g., a file path) of the digital model. The digital model of the asset related to the medical device may be, but is not limited to, a 3-D model file, a virtual reality file, an image file, or a document file. The digital model can include a CAD file of the medical device (or a file path to the CAD file) generated during a design stage of the medical device, providing a virtual representation of the medical device. Where there is a virtual reality file format representation, the virtual reality file or file path to the virtual reality file can also be included.

The associated initial attribute data corresponds to a detail associated with the asset related to the medical device and the ledger file can store the associated initial attribute data with properties providing a record of information about the asset related to the medical device or the detail associated therewith.

Process 300 can further include creating (310) the ledger file for the asset related to the medical device. The ledger file for the asset related to the medical device can be identified by a first object identifier and store the associated initial attribute data and the digital model.

Process 300 can further include generating (315) a first security tag for the digital model, generating (320) a second security tag for the initial attribute data, and updating (325) an index of the ledger file by storing the first security tag and the second security tag. The first security tag can include information of a first block hash to the digital model and a first timestamp. The second security tag can include information of a second block hash to the initial attribute data and a second timestamp.

A security tag can further include what or who is a source of the data or update. In some cases, the source of the data or update is an application or device. In some cases, the source of the data or update indicates the attribute that is included or updated, for example, when the attribute is an application or device.

Referring to FIGS. 4A-4C, a conceptual representation of a ledger file for an asset related to a medical device and a corresponding graphical representation of the ledger file is shown at different points in time (e.g., t₀, t₁, . . . , tₙ), where FIG. 4A shows to, FIG. 4B shows t₁, and FIG. 4C shows to.

In the illustrative example of FIG. 4A, at to, a ledger file (shown as EOX file 405A) is created for the asset related to the medical device. In the illustrative scenario, the EOX file 405A is given a unique identifier (ObjectId) 420 and the content includes a 3D model file 422 (e.g., CAD file) and a virtual reality (XR) model file 424.

The attributes section 426A of the EOX file 405A includes a product information attribute 428, a material attribute 430, an amount attribute 432, and a source attribute 434. Here, the product information attribute 428 is a parent attribute indicative of a category of information; and the material attribute 430, the amount attribute 432, and the source attribute 434 are child attributes of the product information attribute 428 and are indicative of an entity in the category of information.

Graphical representation 410A shows a graphical representation of the ledger file at to (EOX file 405A). In the illustrative scenario, root node 438 represents the medical device, node 440 represents the product information attribute 428, node 442 represents the material attribute 430, node 444 represents the amount attribute 432, and node 446 represents the source attribute 434. Here, node 440 is connected to the root node 438, node 442 is connected to node 440, and node 444 and node 446 are connected to node 442.

As explained above, the EOX file can accumulate data about the asset related to the medical device (i.e., as things occur in the real world, the virtual/digital asset is updated to reflect those things). In this case, additional attributes are added to the EOX file. As previously described, an attribute for a medical device corresponds to a detail associated with the asset related to the medical device. The association may be based on a direct relationship, for example, based on any form of information about the asset related to the medical device. In some cases, a loose association of attributed data can be included, allowing for additional connections between assets. Examples of loosely associated attributes include community information (e.g., information from other similar physical entities or other users who have acquired similar entities), funding and regulation information (e.g., insurance/government regulation), and other aspects that may of interest for filtering and sorting of information. Attributes providing attribution information and even regulatory frameworks/policy information (e.g., information related to but may not be a direct detail) can be part of the metadata of the ledger file as well. For example, with an asset related to a medical device, the attribution information associated with the EOX file can include product information such as manufacturer and regulation/regulatory framework information.

When the EOX file accumulates data about the asset related to the medical device, process 300 can further include receiving second attribute data associated with the asset related to the medical device; generating a third security tag for the second attribute data, the third security tag comprising information of a third block hash to the second attribute data and a third timestamp; and updating the index of the ledger file by storing the third security tag. This process repeats as data is accumulated.

Referring to FIG. 4B, at t₁, additional attribute information has been received reflecting events that have occurred in the real world and the ledger file (shown as EOX file 405B) is updated. In the illustrative scenario, the attributes section 426B of the EOX file 405B includes additional attributes such as a manufacturer information attribute 448, a company attribute 450, a date attribute 452, and a location attribute 454. Here, the manufacturer information attribute 448 is a parent attribute indicative of a category of information; and the company attribute 450, the date attribute 452, and the location attribute 454 are child attributes of the manufacturer information attribute 448 and are indicative of an entity in the category of information.

Graphical representation 410B shows a graphical representation of the ledger file at t₁ (EOX file 405B). In the illustrative scenario, node 456 represents the manufacturer information attribute 448, node 458 represents the company attribute 450, node 460 represents the date attribute 452, and node 462 represents the location attribute 454. Here, node 456 is connected to the root node 438, node 458 and node 460 are connected to node 456, and node 462 is connected to node 458.

Referring to FIG. 4C, at tₙ, additional attribute information has again been received reflecting events that have occurred in the real world and the ledger file (shown as EOX file 405C) has been updated. In the illustrative scenario, the attributes section 426C of the EOX file 405C includes additional attributes such as a sales information attribute 464, a hospital attribute 466, a location attribute 468, a surgeon attribute 470, and a date attribute 472. Here, the sales information attribute 464 is a parent attribute indicative of a category of information; and the hospital attribute 466, the surgeon attribute 470, and the date attribute 472 are child attributes of the sales information attribute 464 and are indicative of an entity in the category of information.

Graphical representation 410C shows a graphical representation of the ledger file at tₙ (EOX file 405C). In the illustrative scenario, node 474 represents the sales information attribute 464, node 476 represents the hospital attribute 466, node 478 represents the surgeon attribute 470, node 480 represents the date attribute 472, and node 482 represents the location attribute 468. Here, node 474 is connected to the root node 438, node 476, node 478 and node 480 are connected to node 474, and node 482 is connected to node 476.

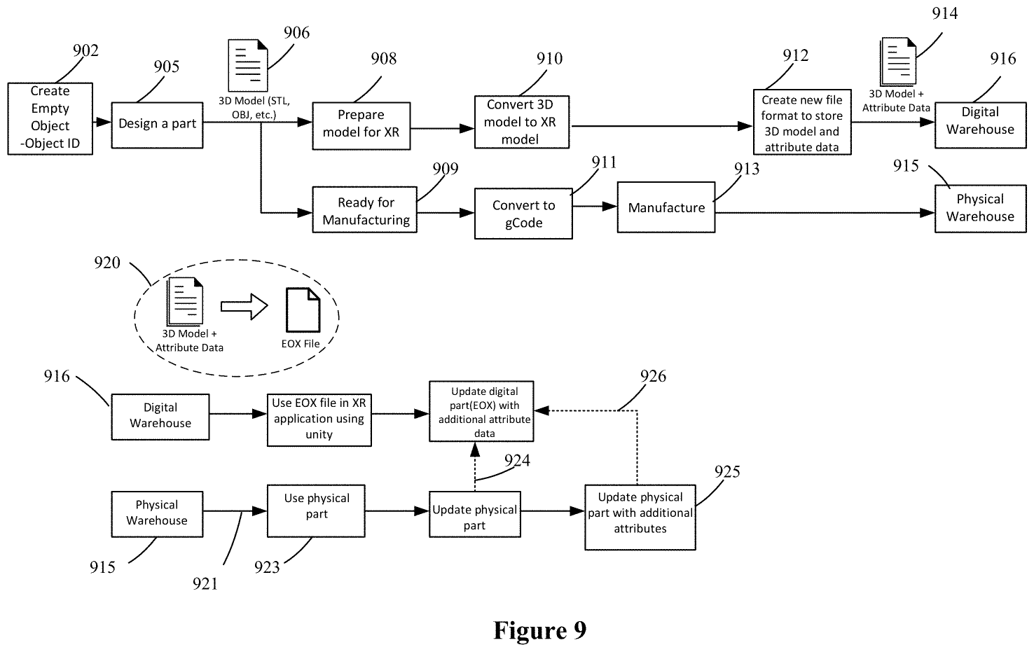

A specific implementation following process 300 is shown and described with respect to FIG. 9.

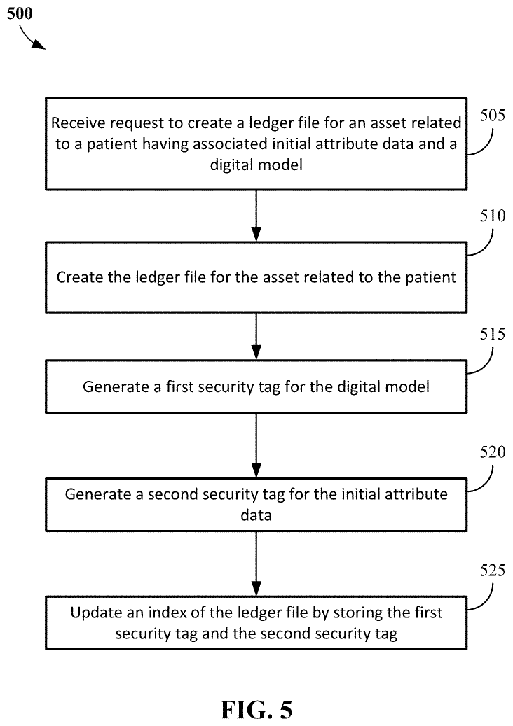

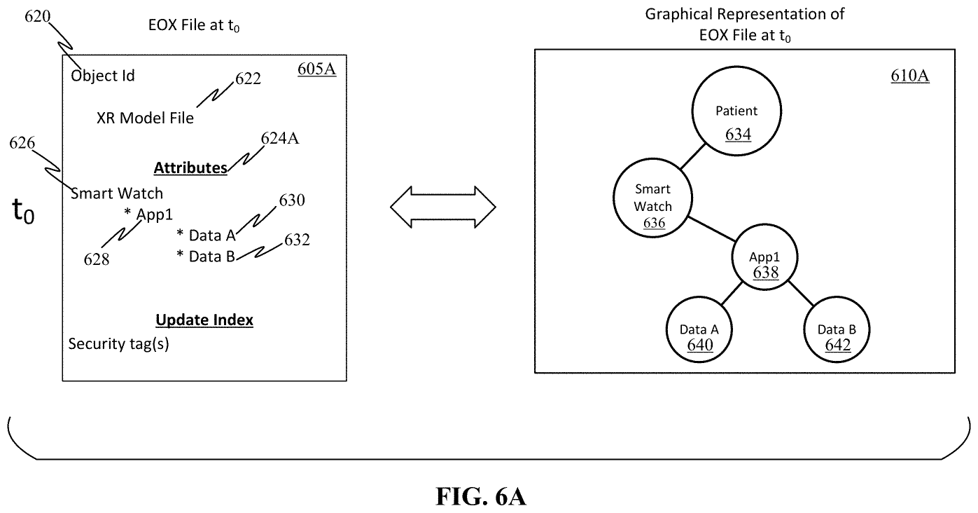

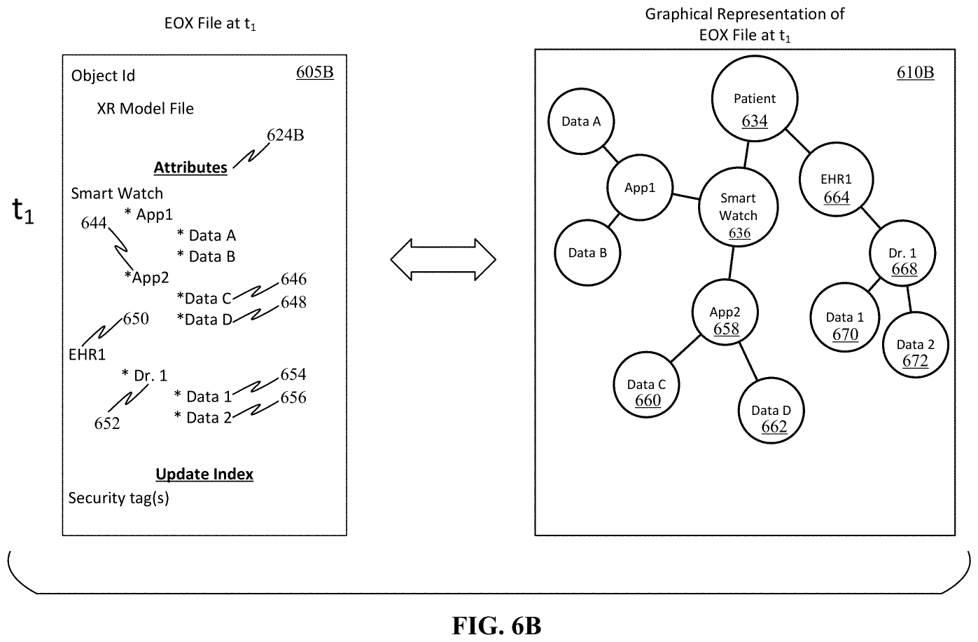

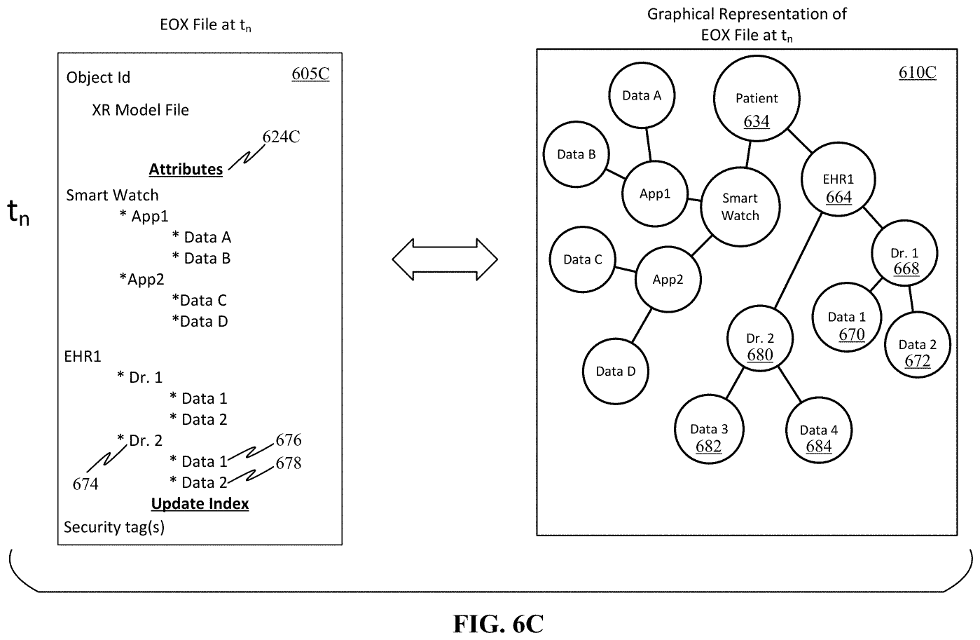

FIG. 5 illustrates an example process flow for creating and updating a ledger file for an asset related to a patient according to certain embodiments of the invention; and FIGS. 6A-6C illustrate an example scenario of creating and updating a ledger file for an asset related to a patient.

As mentioned above, the described EOX file provides a way for digital assets to track various attributes it represents in the physical world. In the illustrative examples of FIG. 5 and FIGS. 6A-6C, the asset is related to a patient. For example, the asset can be a person which will be represented as an avatar and the physical entity can be a specific person.

As the patient performs day-to-day activities, an EOX file can act as a ledger to track any changes to the attributes and also maintain the validity of the changes using concepts from the blockchain.

Some or all of process 500 may be executed at, for example, one or more servers as part of services (e.g., a server may include instructions to perform process 500. In some cases, process 500 may be executed at a computing device while in communication with one or more servers.

Referring to FIG. 5, process 500 can include receiving (505) a request to create a ledger file for an asset related to a patient. The asset related to the patient includes associated initial attribute data and a digital model. For example, the asset can be a person which will be represented as an avatar and the physical entity can be a specific person. In this case, the digital model is a 3D model file and/or a virtual reality (XR) file of the specific person used to generate the avatar of the specific person. In some cases, the request to create the ledger file can be a request to onboard a patient to a healthcare system.

Process 500 can further include creating (510) the ledger file for the asset related to the patient. The ledger file for the asset related to the patient can be identified by a first object identifier and can store the associated initial attribute data and the digital model. The associated initial attribute data can correspond to a detail associated with the asset related to the patient. The ledger file can store the associated initial attribute data with properties providing a record of information about the asset related to the patient or the detail associated therewith.

Process 500 can further include generating (515) a first security tag for the digital model, generating (520) a second security tag for the initial attribute data, and updating (525) an index of the ledger file by storing the first security tag and the second security tag. The first security tag can include information of a first block hash to the digital model and a first timestamp; and the second security tag can include information of a second block hash to the initial attribute data and a second timestamp.

A security tag can further include what or who is a source of the data or update. In some cases, the source of the data or update is an application or device. In some cases, the source of the data or update indicates the attribute that is included or updated, for example, when the attribute is an application or device.

Referring to FIGS. 6A-6C, a conceptual representation of a ledger file for an asset related to a patient and a corresponding graphical representation of the ledger file is shown at different points in time (e.g., t₀, t₁, . . . , tₙ), where FIG. 6A shows to, FIG. 6B shows t₁, and FIG. 6C shows to.

In the illustrative example of FIG. 6A, at t₀, a ledger file (shown as EOX file 605A) is created for the asset related to the patient. In the illustrative scenario, the EOX file 605A is given a unique identifier (ObjectId) 620 and the content includes a virtual reality (XR) model file 622.

There can be any number of categories and subcategories of attributes and properties that are tracked. An attribute corresponds to a detail associated with the asset related to the patient. The attributes can include user input including patient inputs that are added as attributes with associated security tags in the ledger file of the patient. Other information that can contribute data tracked by the ledger file for the asset related to the patient include, but are not limited to, wearable devices (e.g., smart watches), medical devices/sensors (e.g., glucose monitor, thermometer, blood pressure, etc.), medical equipment, and EHR information.

In the illustrative scenario, the attributes section 624A of the EOX file 605A includes a smart watch attribute 626, an application) attribute 628, a data A attribute 630, and a data B attribute 632. Here, the smart watch attribute 626 is a parent attribute indicating a source of the information; and the application 1 attribute 628, the data A attribute 630, and the data B attribute 632 are child attributes of the smart watch attribute 626 and are related to content associated with the source of the information.

Graphical representation 610A shows a graphical representation of the ledger file at to (EOX file 605A). The first block of the (asset related to the) patient (which can be a virtual/digital representation of the patient) becomes the node of aggregation (root node) and the attributes become hierarchical (e.g., depending from or based off of the asset related to the patient). In the illustrative scenario, root node 634 represents the patient, node 636 represents the smart watch attribute 626, node 638 represents application 1 attribute 628, node 640 represents the data A attribute 630, and node 642 represents the data B attribute 632. Here, node 636 is connected to the root node 634, node 638 is connected to node 636, and node 640 and node 642 are connected to node 638.

Returning to FIG. 5, as explained above, the EOX file can accumulate data about the asset related to the patient (i.e., as things occur in the real world, the virtual/digital asset is updated to reflect those things). In this case, additional attributes are added to the EOX file. When the EOX file accumulates data about the asset related to the patient, process 500 can further include receiving second attribute data associated with the asset related to the patient; generating a third security tag for the second attribute data, the third security tag comprising information of a third block hash to the second attribute data and a third timestamp; and updating the index of the ledger file by storing the third security tag.

Referring to FIG. 6B, at t₁, additional attribute information has been received reflecting events that have occurred in the real world and the ledger file (shown as EOX file 605B) has been updated. In the illustrative scenario, the attributes section 624B of the EOX file 605B includes additional attributes such as an application2 attribute 644, a data C attribute 646, and a data D attribute 648, an EHR1 attribute 650, a doctor1 attribute 652, a data1 attribute 654, and a data2 attribute 656. Here, the application2 attribute 644, the data C attribute 646, and the data D attribute 648 are child attributes of the smart watch attribute 626 and are related to content associated with the source of the information. The EHR1 attribute 650 is a parent attribute indicating a source of the information; and the doctor1 attribute 652, the data 1 attribute 654, and the data 2 attribute 656 are child attributes of the EHR1 attribute 650 and are related to content associated with the source of the information.

Graphical representation 610B shows a graphical representation of the ledger file at t₁ (EOX file 605B). In the illustrative scenario, node 658 represents the application2 attribute 644, node 660 represents the data C attribute 646, node 662 represents the data D attribute 648, node 664 represents the EHR1 attribute 650, node 668 represents the doctor1 attribute 652, node 670 represents the data 1 attribute 654, and node 672 represents the data 2 attribute 656. Here, node 658 is connected to node 636, node 660 and node 662 are connected to node 658. Node 664 is connected to the root node 634, node 668 is connected to node 664, and node 670 and node 672 are connected to node 668.

Referring to FIG. 6C, at tₙ, additional attribute information has again been received reflecting events that have occurred in the real world and the ledger file (shown as EOX file 605C) has been updated. In the illustrative scenario, the attributes section 624C of the EOX file 605C includes additional attributes such as a doctor 2 attribute 674, a data 3 attribute 676, and a data 4 attribute 678. Here, the doctor 2 attribute 674, the data 3 attribute 676, and the data 4 attribute 678 are child attributes of the EHR1 attribute 650 and are related to content associated with the source of the information.

Graphical representation 610C shows a graphical representation of the ledger file at tₙ (EOX file 605C). In the illustrative scenario, node 680 represents the doctor 2 attribute 674, node 682 represents the data 3 attribute 676, and node 684 represents the data 4 attribute 678. Here, node 680 is connected to node 664 and node 682 and node 684 are connected to node 680.

As mentioned above, two physical entities having corresponding ledger files may be connected (e.g., a patient having a medical device implant).

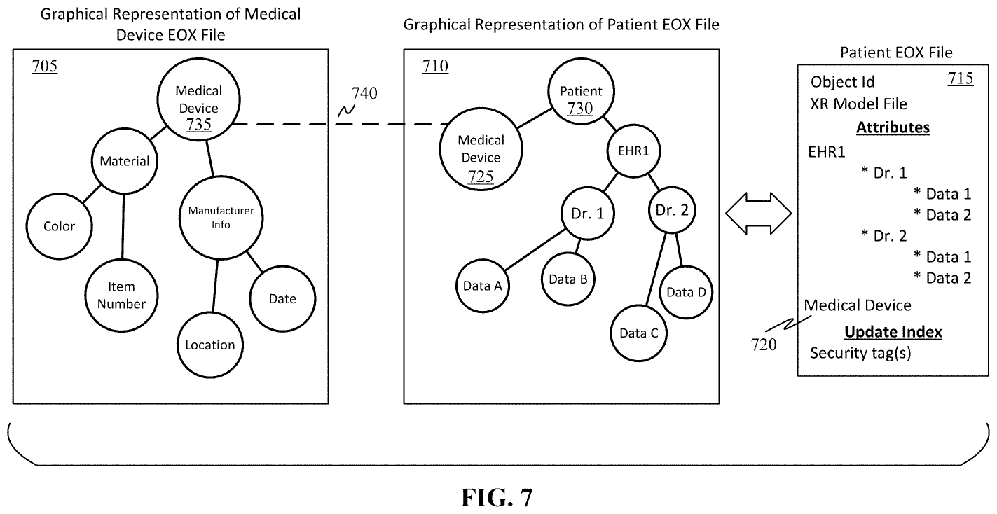

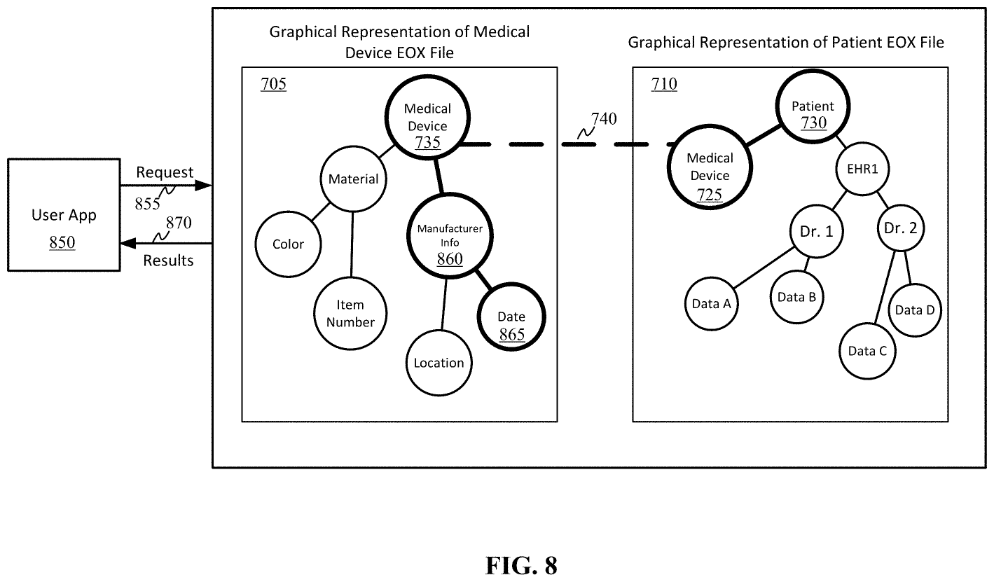

FIG. 7 and FIG. 8 illustrate example scenarios relating to a patient and a medical device according to certain embodiments of the invention where FIG. 7 illustrates an example scenario of updating a ledger file of an asset related to a patient to include the medical device after the medical device has been implanted in the patient and FIG. 8 illustrates an example scenario of accessing data associated with the medical device. Ledger files can be created for assets related to a medical device and a patient (“medical device ledger file” and “patient ledger file”), for example, as described with respect to FIGS. 3-6C. The medical device can be, for example, an implantable medical device such as an artificial knee joint and the patient can be, for example, a patient of a hospital who needs knee replacement surgery.

Referring to FIG. 7 and FIG. 8, a graphical representation of a medical device ledger file is shown as graphical representation 705, a graphical representation of a patient ledger file is shown as graphical representation 710, and a conceptual representation of the patient ledger file is shown as patient EOX file 715.

In the illustrative example of FIG. 7, a medical device is implanted in the patient and the patient ledger file is updated to include reference to the medical device. Here, a medical device attribute 720 is added to the patient EOX file 715 as an attribute, and a medical device attribute node 725 is added to graphical representation 710 as a connection to a patient root node 730. The medical device attribute 720 can indicate a location of information associated with the medical device. When the medical device attribute node 725 is added to graphical representation 710, a link is created between the patient ledger file and the medical device ledger file. This link is represented by a dotted line 740 connecting the medical device attribute node 725 in graphical representation 710 to a medical device root node 735 in the graphical representation 705.

In the illustrative example of FIG. 8, a doctor for the patient needs information regarding the date the medical device was manufactured and uses user application 850 to request the information regarding the medical device from the patient ledger file, for example in accordance with process 250 described with respect to FIG. 2B. Here, the request (e.g., 855) to retrieve the particular information of the medical device can include an object identifier of the patient ledger file and a search query. The search query can be about the manufacture date of the medical device. For the most recent information, the timestamp information of the security tag can be used.

To access the information regarding the medical device, as shown in the conceptual representation, nodes from the graphical representation 705 and the graphical representation 710 are traversed. Here, the path that is traversed includes the patient root node 730, the medical device attribute node 725, medical device root node 735, a manufacturing attribute node 860, and a date attribute node 865. The retrieved information regarding the medical device (results 870) can then be provided to the doctor and displayed on the user application 850.750. It should be understood that the graph representation is for illustrative purposes and the mechanism may be different. For example, as another conceptual representation, the traversal can be considered as being through blocks instead of nodes

An example of the scenarios relating to the patient and the medical device described in FIG. 7 and FIG. 8 can be illustrated by continuing the process 500 of FIG. 5 (the process flow for creating and updating a ledger file of an asset related to a patient). In the case where the patient ledger file is updated after the medical device has been included, process 500 can further include receiving second attribute data associated with the patient, the second attribute data indicating a relationship between the patient and a medical device; generating a third security tag for the second attribute data, the third security tag for the second attribute data comprising information of a third block hash to the second attribute data and a third timestamp; and updating the index of the patient ledger file by storing the third security tag for the second attribute data, wherein updating the index of the patient ledger file provides a link between the medical device and the patient.

Then, with reference to process 250 of FIG. 2B, where information regarding the medical device is requested from the patient ledger file, the process 250 can include receiving a request to retrieve particular information of the medical device, the request comprising the first object identifier assigned to the patient ledger file and an attribute of interest of the medical device; identifying a security tag of an update related to attribute of interest of the medical device; using the security tag to retrieve the particular information of the medical device from an appropriate block; and providing the particular information of the medical device.

The following example scenarios illustrate further applications in which it is possible to provide a virtual asset that has a means to record data of the physical entity's development, sale, and use.

FIG. 9 illustrates a correlation between a virtual and physical entity during attribution of Object ID while the entity moves from origin to end of life according to certain embodiments of the invention. Referring to FIG. 9, an EOX file may be generated for a medical device. Initially, at origin 902, an empty EOX file is created, which is an empty ledger file that is assigned a unique object identifier (Object ID). In the physical world a part is designed (905). During the design process, many different types of attributes can be added to the EOX file, for example, design concept, project charter, business plan/purpose, volatile organic compounds (VOC), design history files (DHF), physical part design using CAD (e.g., 3D model 906 from CAD system, testing, stakeholder approval, regulatory review. All of this information can be entered into the EOX file and assigned corresponding security tags (which can then be used to retrieve the information). For example, the 3D model 906 and the various attribute data can be stored in the EOX file format (912). The 3D model 906 is convertible into XR space, for example the 3D model can be prepared for virtual reality file format (908) and converted to an XR model (910). This XR model can also be stored in the EOX file format (912).

In the physical world, the designed part (e.g., as indicated by the CAD drawings) may be ready for manufacturing (909), converted to gCode (911), and manufactured (913). During this manufacturing process, many different types of attributes can be added to the EOX file, for example, form, material, device master record (DMR), Manufacturing Transfer; Manufacturing Process; Physical Part assigned gCode, machine, tools, contact materials; Manufacturing post processing, packaging, and moved to storage location (e.g., the physical part is post processed, packaged, and put into temporary queue with lot number and serial number and relevant part identification information).

Thus, the attributes/metadata are linked to the CAD model (914) as part of the EOX file (920). The digital information can be stored at a Digital Warehouse 916, assigning timestamp and version (lot number and serial number and relevant part identification information); the physical part may be moved to a physical warehouse 915.

As part of the continued life cycle tracking, it is possible that the physical part is moved (921) from the physical warehouse 915. A recording version and timestamp can be stored in the digital file indicating the movement of the physical part. The physical part can be used (923), for example, for testing, training, sales, demonstration, etc. and the information of use can be recorded as updates in the EOX file (924). This may occur synchronously (if from a connected device) or otherwise input. The EOX file can move in and out of the digital warehouse 916 and be updated (926) each time the physical asset is updated with additional attributes (925).

The EOX file and attributes can track a life cycle of a physical entity and include, over time, the following: an empty Object/Block with a Unique Object ID, which is the starting element of the asset related to the physical entity, similar to block in block chain; and metadata/attributes (e.g., Attributes of Form, Value, Access to profiles, objects, and/or environments; and ownership).

For the attributes of form, a ledger file generator creates subclasses that relate to size, shape, solidity and adoption of physical attributes including replicating physics and physical properties or creation of new physics and physical properties that are unique and not related to natural physics or physical properties.

For the attributes of value, a ledger file generator creates subclasses that include material, energy, transfer method or rate, number, worth, gravity, magnetic properties, entropy, relation to time.

For the attributes of authorization, profiles can be included, along with permission attributes that enable access to searchable data by people, programs, peripherals, audio tools, haptic tools, manufacturing operations. In addition, authentication can be stratified to provide information based on what has been allowed by the higher-level object owner.

For the objects, the attributes can include solid, liquid, gas, transfer mechanisms, and virtual reality construct tools. Other attributes can be included such as loose attributions to related objects, interaction permissions to objects, and transfer of information/imprinting on respective objects.

For the attributes of environments, information of use in various environments and permission to access environments can be included. In addition, Object ID's physical interaction in various environments, and permission to change or alter an Object ID's attributes in various environments, along with transfer of information/imprinting on respective environments can be included.

Attributes can further include selective anonymization—attribute accessibility defined by profile and environment. Selective anonymization can be driven by: Regulation, User/Key Stakeholder, and Environment. The attributes can also further include generalized form/brand for object transfer/maturation/promotion/testing/assignment.

Additional metadata/attributes involve product form, manufacturing, and validation. Examples include assignment of process related data (Design History File/Design Manufacturing Report) such as conception process, manufacturing process, testing process, cost, and time.

Logistics, Sales, Material Source, and other options may be stored as attributes/properties.

Creation of ports/points of access of subject matter experts during process as applicable based on value added to the respective process. Permissions are created based on criticality of information added during respective processes.

Yet further metadata can include information that is organized and presented to stakeholders based on what is import to the stakeholder, a Surgeon/Primary sale target/installer/user Training, simulated surgical use in VR and AR applications, credentials, relation to industry, industry awards, clubs papers, patents, reimbursements, warranties.

The described EOX file can be used on a computing device for simulated use, for example, but not limited to, simulated use in medical (e.g., surgical), industrial, sport, gaming, retail, and military applications.

Projected use with respect to tracking, time, cost, performance, subjective satisfaction, use with associated tools, environments, location, and physiologic attributes. Metrics related to use can include time, cost, and satisfaction. Metadata can further include information regarding any upgrades applied to the asset. Information regarding any ancillary products used in conjunction with the Object ID can be stored and tracked.

In some cases, a loose association of attributed data can be included, allowing for additional connections between assets and/or entities. Examples of loosely associated attributes include information regarding family of products/options and asset compatibility to peripherals. Information regarding family of products/options can be used, for example, in cross promotion of the asset; and information regarding asset compatibility to peripherals can be used in, for example, security analysis as well as risk of misuse/interpretation analysis.

Additional metadata/attributes can include information regarding version history of the entity.

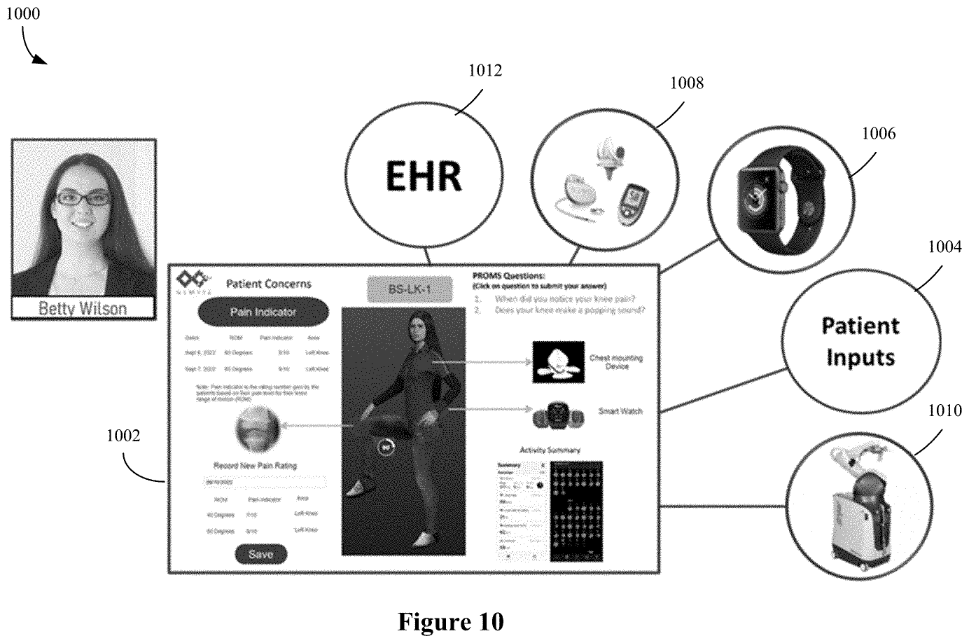

FIG. 10 illustrates an example patient care platform with a ledger file integration in accordance with the subject disclosure. Referring to FIG. 10, a patent care platform 1000 can have a graphical user interface (GUI) 1002 through which a digital model of the patient and corresponding information from a ledger file of the patient can be displayed and manipulated. The GUI 1002 can also enable direct user input including patient inputs 1004 that are added as attributes with associated security tags in the ledger file of the patient. Other information that can contribute data tracked by the ledger file of the patient include, but are not limited to, wearable devices 1006 (e.g., smart watches), medical devices/sensors 1008 (e.g., glucose monitor, thermometer, blood pressure, etc.), medical equipment 1010, and EHR information 1012. Through the described systems that generate and store ledger files, it is possible to integrate medical data and user inputs into an individualized patient experience.

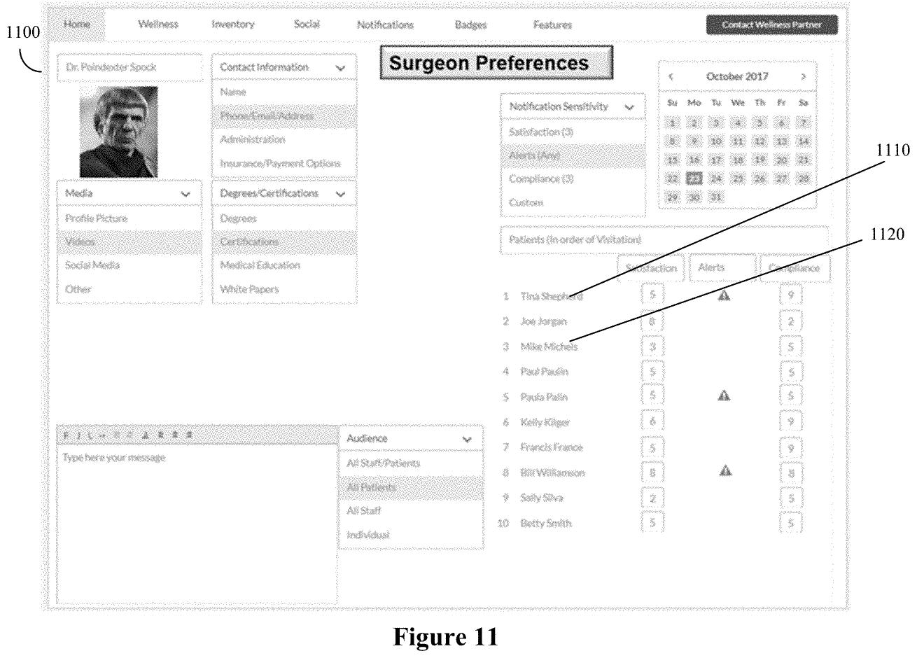

FIG. 11 illustrates an example interface of a patient care platform displaying a surgeon dashboard. Referring to FIG. 11, a surgeon dashboard 1100 can be provided by a patient care platform or as part of an existing electronic health record system (e.g., of a particular hospital system). Any suitable functionality can be provided as part of the surgeon dashboard 1100. Instead of access to one patient's data, the surgeon dashboard 1100 enables a user to access multiple patents' data, including the ledger files of the different patients. For example, the user can select a first patient 1110 or a second patient 1120 and access (or generate) the ledger file for that patient. Here, when the surgeon requests access to information of a patient (e.g., Betty Smith 1130), even though the dashboard is the surgeon's dashboard, the root node/ledger file's digital model is of the selected patient.

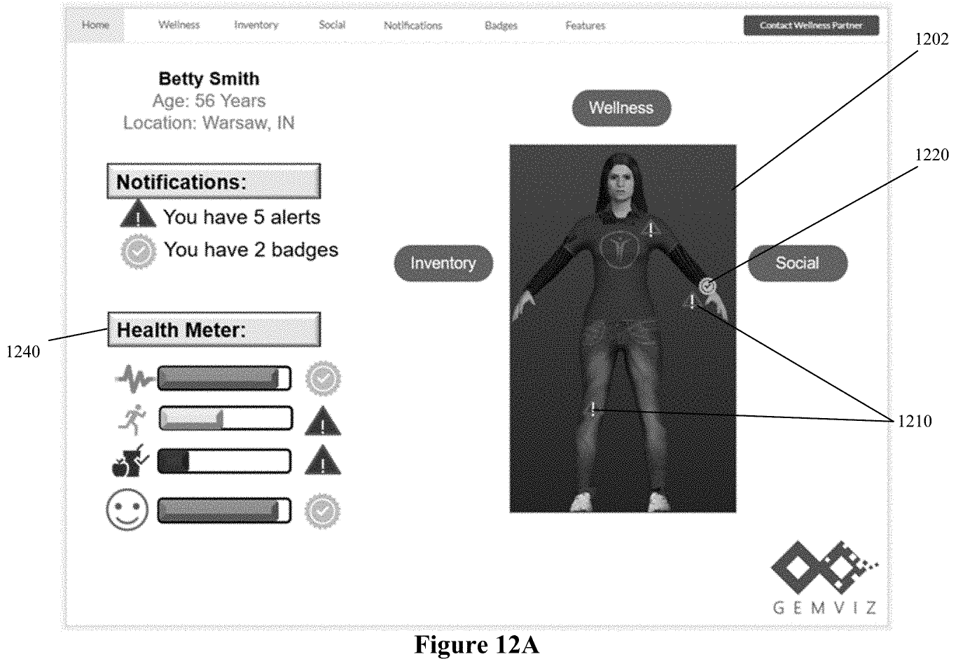

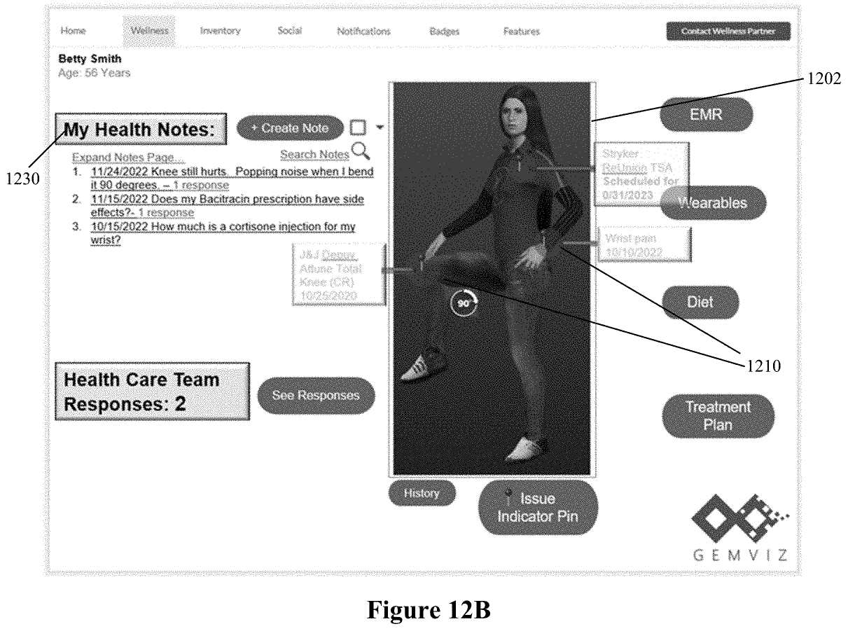

FIGS. 12A and 12B illustrate example interfaces of a patient care platform displaying a selected patient view. Referring to FIGS. 12A and 12B, upon selection of a particular patient (e.g., Betty Smith 1130 via surgeon dashboard 1100 of FIG. 11 or by the patient logging in), the patient's avatar 1202 (e.g., from the digital model) can be displayed, along with information of various attributes of the patient.

As can be seen, the first asset where data is attributed to is related to a particular patient. The patient becomes the node of aggregation and the attributes become hierarchical (based off of the patient). There can be any number of categories and subcategories of attributes and properties that are tracked. The patient care platform can both input data to the ledger file and request data from the ledger file, which displays in part on the avatar of the patient. For example, alerts 1210 may be provided (e.g., as indicated in retrieved data and optionally displayed as part of the avatar). In addition, information from a wearable (e.g., smart watch 1220 shown in FIG. 12A) can be accessed by interacting with the digital representation of that attribute associated with the patient's ledger file.

Similarly, referring to FIG. 12B, health notes 1230 can be considered a category attribute in the ledger file of the patient with content of user (patient or health practitioner) input. When a particular note is initially input, the health notes attribute 1230 is updated and a security tag is assigned. The timestamp of the security tag can be used to indicate the note date.

Referring to FIG. 12A with the security tags and data that are built into the category and subcategory structures, it is possible to aggregate the information and generate metrics (e.g., for health aspects 1240). For example, based on a search parameter, results of the search (for retrieving particular information from the ledger file) can be evaluated for metrics (e.g., identifying how many complaints over a period of time or for a certain body part).

Certain Embodiments may be implemented as a computer process, a computing system, or as an article of manufacture, such as a computer program product or computer-readable storage medium. Certain methods and processes described herein can be embodied as software, code and/or data, which may be stored on one or more storage media. Certain embodiments of the invention contemplate the use of a machine in the form of a computer system within which a set of instructions, when executed by hardware of the computer system (e.g., a processor or processing system), can cause the system to perform any one or more of the methodologies discussed above. Certain computer program products may be one or more computer-readable storage media readable by a computer system (and executable by a processing system) and encoding a computer program of instructions for executing a computer process. It should be understood that as used herein, in no case do the terms “storage media”, “computer-readable storage media” or “computer-readable storage medium” consist of transitory carrier waves or propagating signals.

Computer systems described herein may be embodied as one or more blade server devices, standalone server devices, personal computers, virtual reality headsets, routers, hubs, switches, bridges, firewall devices, intrusion detection devices, mainframe computers, network-attached storage devices, and other types of computing devices, and combinations thereof. Such systems include one or more hardware processors and one or more computer readable storage media.

It should be understood that the examples and embodiments described herein are for illustrative purposes only and that various modifications or changes in light thereof will be suggested to persons skilled in the art and are to be included within the spirit and purview of this application.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts that would be recognized by one skilled in the art are intended to be within the scope of the claims.

What is claimed is:

1. A computer-implemented method comprising:
receiving, by a computing system, a request to create a ledger file for an asset related to a medical device and having associated initial attribute data and a digital model;
creating, by the computing system, the ledger file for the asset related to the medical device, the ledger file being identified by a first object identifier and storing the associated initial attribute data and the digital model;
generating, by the computing system, a first security tag for the digital model, the first security tag comprising information of a first block hash to the digital model and a first timestamp;
generating, by the computing system, a second security tag for the initial attribute data, the second security tag comprising information of a second block hash to the initial attribute data and a second timestamp; and
updating, by the computing system, an index of the ledger file by storing the first security tag and the second security tag.

2. The computer-implemented method of claim 1, wherein the associated initial attribute data corresponds to a detail associated with the asset related to the medical device, wherein the ledger file stores the associated initial attribute data with properties providing a record of information about the asset related to the medical device or the detail associated therewith.

3. The computer-implemented method of claim 1, further comprising:
receiving second attribute data associated with the asset related to the medical device;
generating a third security tag for the second attribute data, the third security tag comprising information of a third block hash to the second attribute data and a third timestamp; and
updating the index of the ledger file by storing the third security tag.

4. The computer-implemented method of claim 1, further comprising:
receiving a request to retrieve particular information of the asset related to the medical device, the request comprising the first object identifier assigned to the ledger file for the asset related to the medical device and an attribute of interest;
identifying a security tag of an update related to the attribute of interest; and
using the security tag to retrieve the particular information from an appropriate block.

5. The computer-implemented method of claim 1, wherein the digital model of the asset related to the medical device comprises resource location information of the digital model.

6. The computer-implemented method of claim 1, wherein the digital model of the asset related to the medical device is a 3-D model file, a virtual reality file, an image file, or a document file.

7. The computer-implemented method of claim 1, wherein each security tag further comprises a source.

8. The computer-implemented method of claim 7, wherein the source is an application or a device.

9. A computer readable storage medium having instructions stored thereon that when executed by a hardware processor, direct the hardware processor to at least:
receive a request to create a ledger file for an asset related to a patient and having associated initial attribute data and a digital model;
create the ledger file for the asset relating to the patient, the ledger file being identified by a first object identifier and storing the associated initial attribute data and the digital model, wherein the associated initial attribute data corresponds to a detail associated with the asset related to the patient, wherein the ledger file stores the associated initial attribute data with properties providing a record of information about the asset related to the patient or the detail associated therewith;
generate a first security tag for the digital model, the first security tag comprising information of a first block hash to the digital model and a first timestamp;
generate a second security tag for the initial attribute data, the second security tag comprising information of a second block hash to the initial attribute data and a second timestamp; and
update an index of the ledger file by storing the first security tag and the second security tag.

10. The computer readable storage medium of claim 9, wherein the instructions further direct the hardware processor to at least:
receive second attribute data associated with the asset related to the patient;
generate a third security tag for the second attribute data, the third security tag comprising information of a third block hash to the second attribute data and a third timestamp; and
update the index of the ledger file by storing the third security tag.

11. The computer readable storage medium of claim 9, wherein the instructions further direct the hardware processor to at least:
receive second attribute data associated with the patient, the second attribute data indicating a relationship between the patient and a medical device;
generate a third security tag for the second attribute data, the third security tag for the second attribute data comprising information of a third block hash to the second attribute data and a third timestamp; and
update the index of the ledger file by storing the third security tag for the second attribute data, wherein updating the index of the ledger file provides a link between the medical device and the patient.

12. The computer readable storage medium of claim 11, wherein the instructions further direct the hardware processor to at least:
receive a request to retrieve particular information of the medical device, the request comprising the first object identifier assigned to the ledger file for the asset related to the patient and an attribute of interest of the medical device;
identify a security tag of an update related to attribute of interest of the medical device;
use the security tag to retrieve the particular information of the medical device from an appropriate block; and
provide the particular information of the medical device.

13. A system comprising:
one or more computer readable storage media;
one or more processors; and
program instructions stored on the one or more computer readable storage media that, when executed by the one or more processors, direct the system to at least:
receive a request to create a ledger file for an asset related to a first physical entity having associated initial attribute data and a digital model;
create the ledger file for the asset related to the first physical entity, the ledger file being identified by a first object identifier and storing the associated initial attribute data and the digital model, wherein the associated initial attribute data corresponds to a detail associated with the asset related to the first physical entity, wherein the ledger file stores the associated initial attribute data with properties providing a record of information about the asset related to the first physical entity or the detail associated therewith;
generate a first security tag for the digital model, the first security tag comprising information of a first block hash to the digital model and a first timestamp;
generate a second security tag for the initial attribute data, the second security tag comprising information of a second block hash to the initial attribute data and a second timestamp; and
update an index of the ledger file by storing the first security tag and the second security tag.

14. The system of claim 13, further comprising instructions to:
receive second attribute data associated with the asset related to the first physical entity;
generate a third security tag for the second attribute data, the third security tag comprising information of a third block hash to the second attribute data and a third timestamp; and
update the index of the ledger file by storing the third security tag.

15. The system of claim 13, further comprising instructions to:
receive a request to retrieve particular information of the asset related to the first physical entity, the request comprising the first object identifier assigned to the ledger file for the asset related to the first physical entity and an attribute of interest;
identifying a security tag of an update related to the attribute of interest;
using the security tag to retrieve the particular information from an appropriate block; and
provide the particular information of the asset related to the first physical entity.

16. The system of claim 13, further comprising instructions to:
receive a request to create a second ledger file for an asset related to a second physical entity having associated initial second physical entity attribute data and a second physical entity digital model;
create the second ledger file for the asset related to the second physical entity, the second ledger file being identified by a second object identifier and storing the associated initial second physical entity attribute data and the second physical entity digital model, wherein the associated initial second physical entity attribute data corresponds to a detail associated with the asset related to the second physical entity, wherein the second ledger file stores the associated initial second physical entity attribute data with properties providing a record of information about the asset related to the second physical entity or the detail associated therewith;
generate a first security tag for the second physical entity digital model, the first security tag for the second physical entity digital model comprising information of a first block hash to the second physical entity digital model and a first timestamp;
generate a second security tag for the initial second physical entity attribute data, the second security tag comprising information of a second block hash to the initial second physical entity attribute data and a second timestamp; and
update an index of the second ledger file by storing the first security tag for the second physical entity digital model and the second security tag for the initial second physical entity attribute data.

17. The system of claim 16, further comprising instructions to:
receive second attribute data associated with the asset related to the second physical entity, the second attribute data indicating a relationship between the asset related to the first physical entity and the asset related to the second physical entity;
generate a third security tag for the second attribute data associated with the second physical entity, the third security tag for the second attribute data comprising information of a third block hash to the second attribute data and a third timestamp; and
update the index of the second ledger file by storing the third security tag for the second attribute data, wherein updating the index of the second ledger file provides a link between the asset related to the first physical entity and the asset related to the second physical entity.

18. The system of claim 17, further comprising instructions to:
receive a request to retrieve particular information of the asset related to the first physical entity, the request comprising the second object identifier assigned to the second ledger file for the asset related to the second physical entity and an attribute of interest of the asset related to the first physical entity;
identify a security tag of an update related to attribute of interest of the asset related to the first physical entity;
use the security tag to retrieve the particular information of the asset related to the first physical entity from an appropriate block; and
provide the particular information of the asset related to the first physical entity.

19. The system of claim 14, further comprising instructions to:
receive second attribute data associated with the asset related to the first physical entity, the second attribute data indicating a relationship between the asset related to the first physical entity and an asset related to a second physical entity having an associated second ledger file;
generate a third security tag for the second attribute data, the third security tag comprising information of a third block hash to the second attribute data and a third timestamp; and
update the index of the ledger file by storing the third security tag.

20. The system of claim 19, further comprising instructions to:
receive a request to retrieve particular information of the asset related to the second physical entity, the request comprising the first object identifier assigned to the ledger file for the asset related to the first physical object and an attribute of interest of the asset related to the second physical entity;
identifying a security tag of an update related to the attribute of interest of the asset related to the second physical entity;
using the security tag to retrieve the particular information from an appropriate block; and
provide the particular information of the asset related to the second physical entity.
